(19) United States
(12) Patent Application Publication
(10) Pub. No.: US 2009/0281463 A1
(43) Pub. Date: Nov. 12, 2009

(54) THERAPY APPARATUS WITH SEQUENTIAL FUNCTIONING

(75) Inventors: Jean-Yves Chapelon, Villeurbanne (FR); David Melo De Lima, Lyon (FR); Emmanuel Blanc, Saint Genis Laval (FR); Laura Curiel-Ramirez Del Prado, Toronto (CA)

Correspondence Address: DENNISON, SCHULTZ & MACDONALD, 1727 KING STREET, SUITE 105, ALEXANDRIA, VA, 22314, US

(73) Assignee: EDAP S.A., Vaulx-en-Velin (FR)

(21) Appl. No.: 12/306,535

(22) PCT Filed: Jul. 5, 2007
(86) PCT No.: PCT/FR07/51601

§ 371 Date: Apr. 10, 2009

(30) Foreign Application Priority Data
Jul. 5, 2006 (FR) 0652813
Jul. 5, 2006 (FR) 0652816

(51) Int. Cl. A61N 7/00

(52) U.S. Cl. 601/2

(57) ABSTRACT

A therapy apparatus for treating tissues by emission of focused ultrasound waves, includes a probe having a transducer formed by ultrasound elements distributed in several categories (N₁ to N_{M}) on a face for emission of focused ultrasound waves which is a surface of revolution generated by the rotation of a curve segment around an axis of symmetry, with the categories of ultrasound elements define independent and substantially contiguous focal volumes, and a control circuit including a signal generator for independently activating, alternately and substantially consecutively, the ultrasound emitter categories in such a way as to ensure continuous insonification in a ring shape originating from different categories of ultrasound emitters.

The present invention relates to the technical area of high-intensity focused ultrasound (HIFU) and more precisely focuses on therapeutic treatment by such focused ultrasound.

It is known that therapy by focused ultrasound creates biological lesions in tissue resulting from a combination of thermal effects and acoustic cavitation activity. The form of these tissue lesions comes directly from the profile of the applied ultrasound field. Therefore, for example in the case of a transducer of spherical form, tissue lesions are substantially elliptical while being centred on the focal point.

For some applications, there is the need to form tissue lesions in crown form. For example, a tissue lesion in crown form can surround a tumor in order to isolate it, on a vascular plane, and cause its necrosis. Similarly, a lesion in crown form can be made on the periphery of a vein or an artery at the junction site with an organ.

The prior art has proposed various treatment probes by focused ultrasound with a view to obtaining biological lesions in crown form.

The document US 2006/009 753 describes therapy apparatus whereof the treatment probe comprises a cylindrical transducer of ultrasound waves emission focused by means of a reflector of conical shape. Such therapy apparatus is not easy to manufacture. In addition, the focusing obtained with a reflector is less effective than focusing coming directly from the geometry of the transducer.

Similarly, the document “Local hyperthermia with MR-guided focused ultrasound: Spiral trajectory of the focal point-optimized temperature uniformity in the target region” Journal of Magnetic Resonance Imaging 12: 571-583 (published in 2000) describes therapy apparatus comprising a multi-element transducer of spherical geometry. This document describes the creating of a set of biological lesions following a trajectory in the form of a spiral (in this sense approaching a crown) enclosing the volume to be destroyed, such as a tumor for example. This trajectory is adopted with a view to obtaining a more homogeneous thermal dose in the volume to be destroyed. However, such apparatus enables formation of biological lesions in crown form only by repetition and spatial juxtaposition of elementary lesions.

Patent application EP 0 421 290 describes a transducer of spherical shape at the centre of which a plane inactive zone is arranged. The effect of such construction is to modify the focusing profile of the ultrasound waves to obtain, when the diameter of the plane inactive surface is sufficiently large, a focal patch of conical form in which the maximum pressure is always centred on the axis of symmetry of the transducer and not on a peripheral crown.

In an attempt to resolve this problem, patent application EP 0 421 290 proposes cutting a transducer of geometry spherical into several sectors which are spread apart from one another or turned with a certain angulation to separate the focusing points and place them at a distance from the axis of symmetry of the transducers. It is clear that this mechanical construction is relatively complicated to do and difficult to control. In addition, per construction, the number and position of the focal points are fixed, considerably limiting treatment possibilities.

In general, it should be noted that it is always possible to synthesise an annular focal patch from a multi-element transducer of spherical shape since each ultrasound element has a size of less than a demi-wavelength. Since the wavelength is very small relative to the size of the transducer, this solution cannot be employed in practice without attracting a very large number of ultrasound elements and the technical problems and costs associated with this large number of ultrasound elements.

By way of complement, it must be considered that the treated volume depends on different parameters, in particular including the exposure duration and temporal and spatial spacing between the ultrasound shots. When these parameters are correctly defined, the treated volume remains restrained to the focal zone of the emitting surface, allowing this technique to be used as a barely invasive surgery instrument.

For treating larger volumes it is known to shift the focal zone. The first technique consists of mechanically moving the ultrasound emitter between each ultrasound exposure. A second known technique consists of electronically moving the focal zone by exciting the emitters with a delay law.

Currently, it must be considered that treatments by focused ultrasound are to date mainly conducted using pause times between the ultrasound exposures to allow cooling of the tissue and ultrasound emitters. This results in a relatively large increase in the length of treatment. In addition, the cavitation and thermal reheating activity are not maintained in the treatment zone during the pause time, which attenuates the efficacy of the treatment.

In the prior art, it has been proposed to execute continuous ultrasound shots during which the ultrasound emitters and the intermediary tissue are permanently stressed, creating a risk of destruction of the ultrasound emitters and burning of the tissue, such as interface lesions or superficial burns, outside the target zone.

The aim of the invention is thus to rectify the disadvantages of the prior art by proposing novel therapy apparatus conceived to focus ultrasound waves according to a crown and/or in focal volumes distributed according to a crown, the number and position of the focal volumes being able to be regulated simply and non-limiting as a function of the tissue lesion to be produced, the operation of this apparatus maintaining continuous heating of the tissue in the treatment zone, while protecting the tissue located on the acoustic path outside the treatment zone.

To attain such an aim, the object of the invention is to propose therapy apparatus for the treatment of tissue by emission of focused ultrasound waves.

According to the invention, the apparatus comprises:
a probe comprising a transducer formed by ultrasound elements distributed into several categories over an emission face of focused ultrasound waves which is a revolution surface engendered by the rotation of a segment of a curve about an axis of symmetry, the categories of ultrasound elements defining independent and substantially contiguous focal volumes,
and a control circuit comprising a signal generator controlled to activate independently, alternatively and substantially consecutively the categories of ultrasound emitters so as to ensure continuous insonification according to a crown, originating from different categories of ultrasound emitters.

According to a preferred embodiment, the ultrasound elements are distributed over an emission face of focused ultrasound waves having a revolution surface engendered by rotation about an axis of symmetry, of a segment of a concave or convex curve of given length having a centre of curve located at a distance from the axis of symmetry, with R≠0.

According to a preferred variant embodiment, the revolution surface is engendered by a segment of an arc of a circle of given length, of given radius and with a centre located at a distance of the axis of symmetry with this distance≠0.

According to another variant embodiment, the distance of the centre of the segment of an arc of a circle from the axis of symmetry is less than the radius of the arc of a circle.

Advantageously, the revolution surface delimits in its central region an opening centred on the axis of symmetry and adapted to receive an imaging transducer.

According to an advantageous characteristic of the subject matter of the invention, the categories of ultrasound emitters correspond either to concentric rings, or to radial sectors, or to radial groups of ultrasound emitters.

According to a first variant embodiment, the ultrasound emitters are distributed according to concentric rings and in that the control circuit comprises a signal generator controlled to deliver signals to activate the ultrasound emitters of each of said concentric rings, with a delay or phase law for ensuring shifting of the focusing crown according to the focusing axis.

According to another embodiment, the ultrasound emitters are distributed into radial sectors and in that the control circuit comprises a signal generator controlled to deliver signals to activate the ultrasound emitters of each of said radial sectors, with a delay or phase law for ensuring shifting of the focusing crown.

According to a preferred variant embodiment, the ultrasound emitters are distributed into concentric rings, divided into radial sectors and the control circuit comprises a signal generator controlled to deliver signals to activate the ultrasound emitters of each of said radial sectors, with a delay or phase law for ensuring shifting of the focusing crown.

Advantageously, the radial sectors of ultrasound emitters are combined to form radial groups and in that the control circuit comprises a signal generator controlled to deliver signals to activate the ultrasound emitters of each of said radial groups.

The control circuit preferably comprises a signal generator controlled to deliver for each radial group, signals to activate the ultrasound emitters of each of the radial groups, with a delay or phase law.

Advantageously, the control circuit comprises a signal generator controlled to deliver signals to activate according to a sequence which varies or which repeats cyclically, a category of ultrasound emitters corresponding either to a ring, a sector or a radial group of ultrasound emitters.

The control circuit preferably comprises a signal generator controlled to deliver signals to activate assemblies of categories of ultrasound emitters composed either of distinct categories of ultrasound emitters or categories whereof at least some are common to the assemblies.

Other subject matter of the invention focuses on decreasing the number of connection cables of the ultrasound emitters. To this effect, the radial groups form identical assemblies of ultrasound emitters and the control circuit is connected to the ultrasound emitters by means of coaxial cables, whereof on the one hand, the cores of all the ultrasound emitters of the same row in the different radial groups are connected together and, on the other hand, the grounds of all the ultrasound emitters of the same radial group are connected to one another, the signal to activate the ultrasound emitters being converted into ultrasound energy solely by the ultrasound emitters of the radial groups whereof the grounds are physically connected to the ground of the activation signal.

Various other characteristics will emerge from the following description in reference to the attached drawings which show, by way of non-limiting examples, embodiments of the subject matter of the invention.

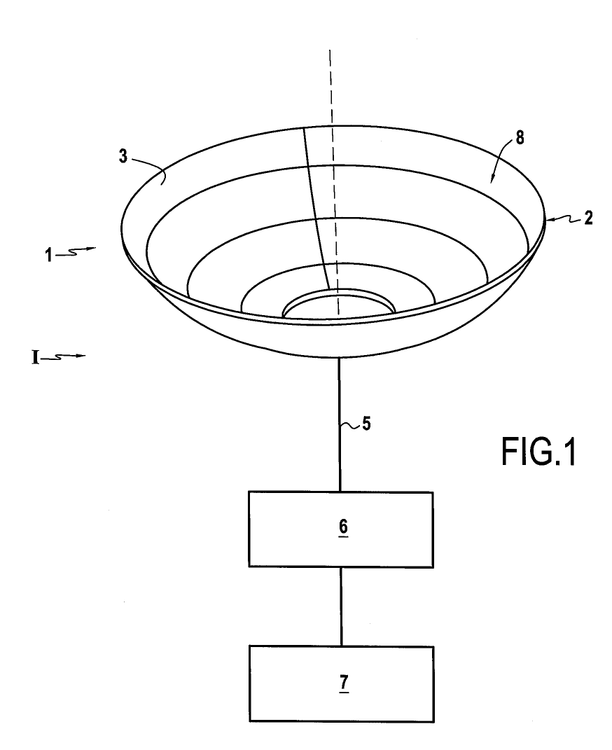

FIG. 1 is a diagram of therapy apparatus in keeping with the invention.

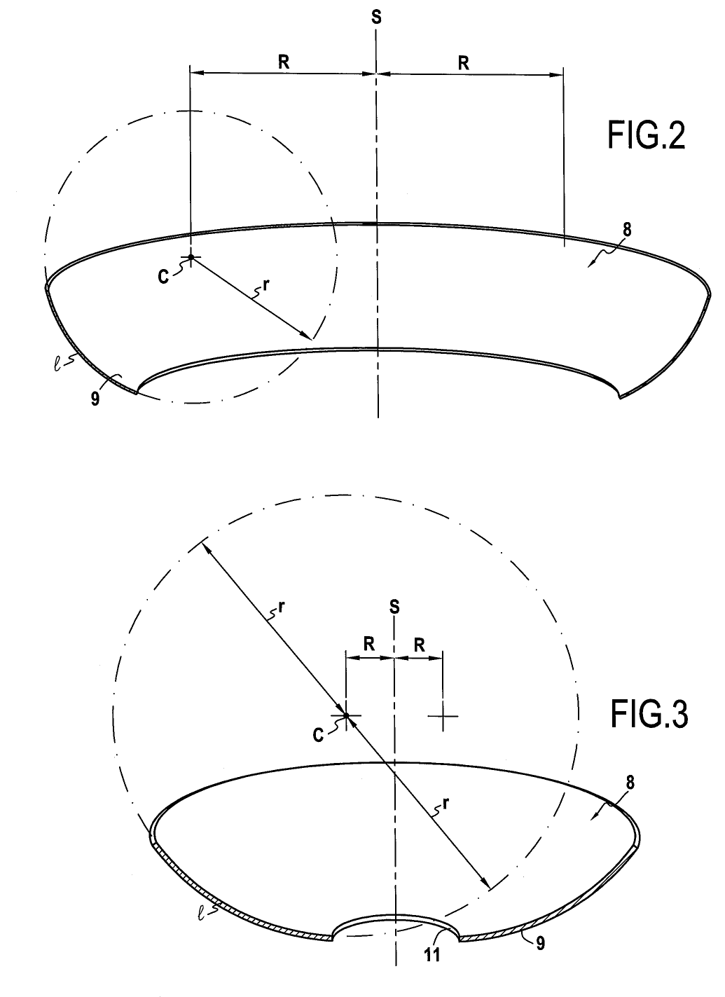

FIGS. 2 to 4 are semi-views of variant embodiments of probes forming part of therapy apparatus in keeping with the invention.

FIGS. 5 to 7 are partial views comprising different organisations of the ultrasound emitters forming part of a therapy probe.

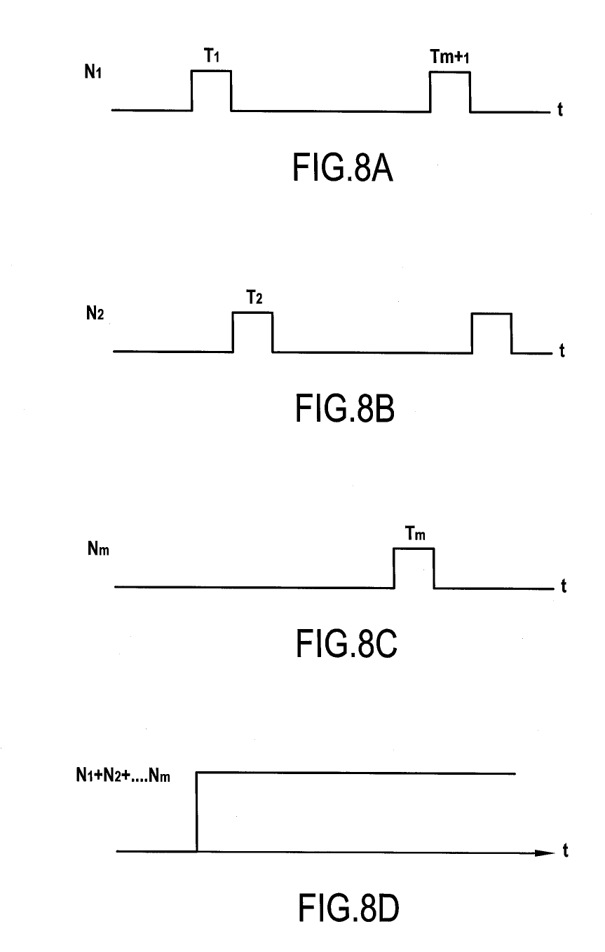

FIGS. 8A to 8C are chronograms illustrating an activation principle as a function of the time of the ultrasound emitters of therapy apparatus in keeping with the invention.

FIG. 8D illustrates over time the treatment obtained in the treatment zone by therapy apparatus in keeping with the invention.

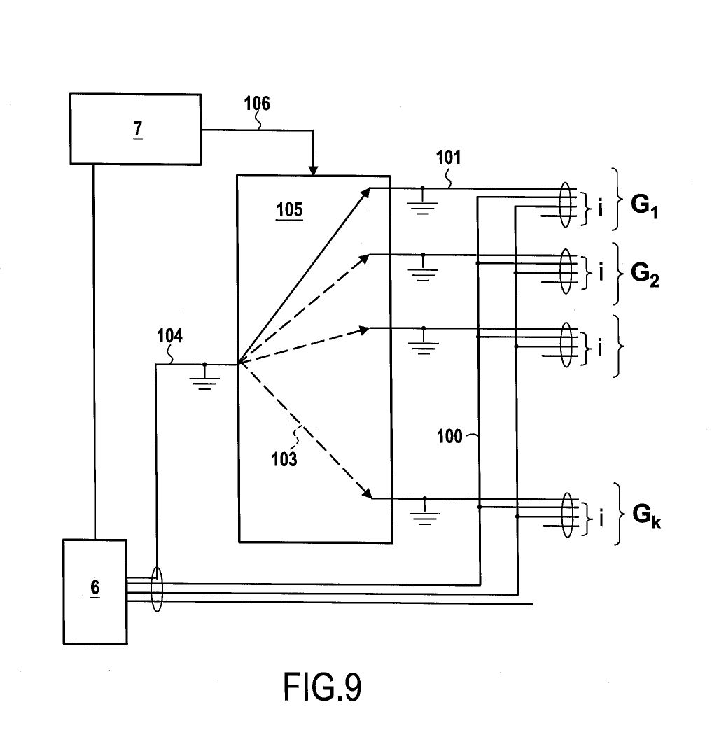

FIG. 9 is a diagram illustrating a preferred connection principle of the ultrasound emitters.

As will emerge more clearly from FIG. 1, the subject matter of the invention relates to therapy apparatus I in the general sense, comprising a therapy probe 1 adapted to carry out treatment on tissue of a living being by means of focused ultrasound high-intensity (HIFU). The therapy probe 1 comprises especially a transducer 2 comprising one or more ultrasound emitters 3 such as for example piezoelectric elements. These ultrasound emitters 3 are connected by means of coaxial cables 5 via an amplifier stage 6 to a control circuit 7 delivering signals to activate the ultrasound emitters 3. The control circuit 7 is not described in greater detail since its manufacture is familiar to the expert. This control circuit 7 accordingly conventionally comprises a controlled signal generator which is connected to the ultrasound emitters by means of the amplifier stage 6.

The transducer 2 has an emission face 8 of focused ultrasound waves. According to a preferred exemplary embodiment illustrated more particularly in FIG. 2, this emission face 8 is a revolution surface engendered by rotation about an axis of symmetry S of a segment of a concave or convex curve 9, of length I and having a centre of curve C located at a distance R from the axis of symmetry S, with R different to zero.

According to a first preferred variant embodiment illustrated in FIG. 2, the revolution surface 9 is engendered by a segment of an arc of a circle of length I, of radius r and with a centre c located at a distance R from the axis of symmetry S, with R different to zero. The geometry of this revolution surface 9 can be considered as coming from toric geometry.

According to a preferred variant embodiment illustrated in FIG. 3, the revolution surface 9 is engendered by a segment of an arc of a circle of centre C, of length I and whereof the distance R of the centre C from the axis of symmetry S is less than the radius r. It can be considered that the geometry comes from geometry relative to a so-called crossed torus. For example, the length I of the segment of an arc of a circle has a value of less than π r/2.

Such as emerges more clearly from FIG. 3, the revolution surface 9 delimits in its central region an opening 11 centred on the axis of symmetry S and having a diameter for example equal to 2·R. Advantageously, this opening 11 is adapted to receive an imaging transducer.

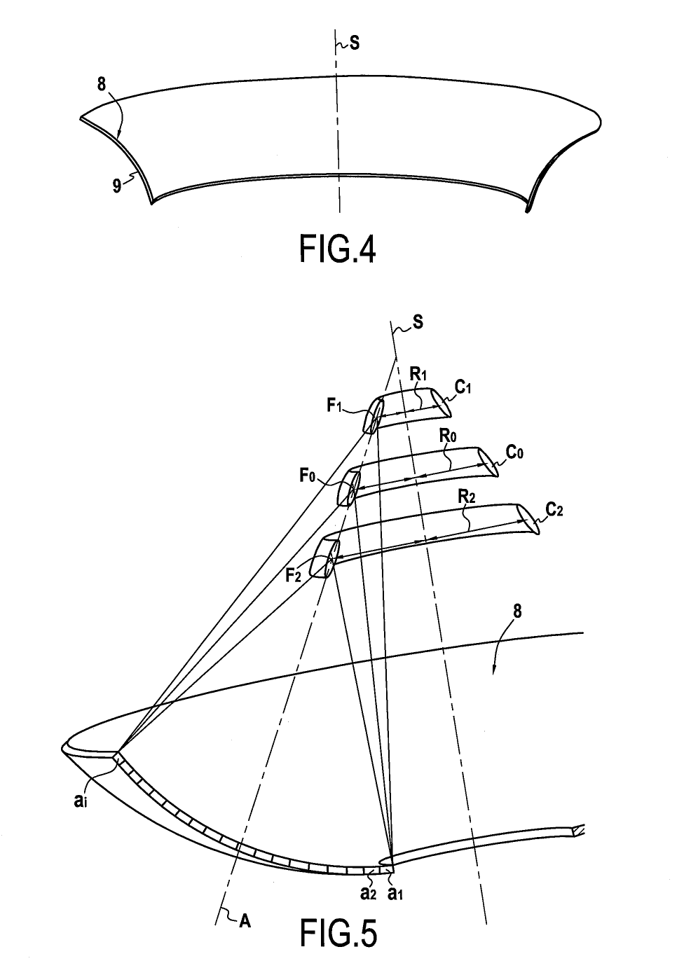

According to the exemplary embodiments illustrated in FIGS. 2 and 3, the revolution surface 9 is engendered by a segment of an axis of a circle whereof the concavity is turned towards the axis of symmetry S. Of course, the revolution surface 9 can be engendered by a segment of an arc of a circle whereof the convexity is turned towards the axis of symmetry S as illustrated in FIG. 4. In the same sense, the revolution surface 9 can be engendered by a segment of a curve different to an arc of a circle. Therefore, the revolution surface 9 can be engendered by a segment of a curve whereof the distance r between each point of the segment of a curve and the centre of curve C has a continuous variation (without inflection point) such as a segment of an elliptical curve, for example.

As will become clear from the above description, the emission face 8 of the transducer is of toric geometry. In general, the emission face 8 of the transducer has a form which is a function of the geometry of the segment of a curve engendering the emitting surface of ultrasounds by rotation about an axis of symmetry, the segment of a curve being able to have various shapes.

According to another characteristic of the invention, the ultrasound emitters 3 are organised on the emission face 8 so as to be distributed into several categories N₁ to N_{M} each defining a point, a patch, a zone or generally a focal volume or a focusing volume. As will become clear from the remainder of the description, the focal volumes are independent and substantially contiguous.

FIG. 5 illustrates an exemplary embodiment in which the ultrasound emitters 3 are distributed into concentric rings a₁ to aᵢ. The transducer 2 according to the invention is thus formed by an annular network of i concentric rings each constituted by an ultrasound emitter 3. These ultrasound emitters 3 are activated by signals delivered by the control circuit 7 without delay or phase law so as to ensure natural focusing of the ultrasound waves according to a crown C₀. Such an arrangement thus ensures natural focusing according to a crown situated at a focal distance F₀ relative to the face 8 by being taken on the focusing axis A. This focusing crown C₀ has a radius R₀.

According to a variant embodiment, the control circuit 7 delivers signals to activate the ultrasound emitters 3 of the concentric rings a₁ to aᵢ; with a delay or phase law for ensuring shifting of the focusing crown according to the focusing axis A. The control circuit 7 thus executes dynamic electronic focusing so as to shift the crown focal from its natural position C₀ to a distal position C₁ and a proximal position C₂. Each focusing crown C₁, C₂ is positioned in the space by the focal distance respectively F₁, F₂ and its radius respectively R₁, R₂. Such configuration adapts to treatment of tumors of different sizes and situated at different depths in the tissue.

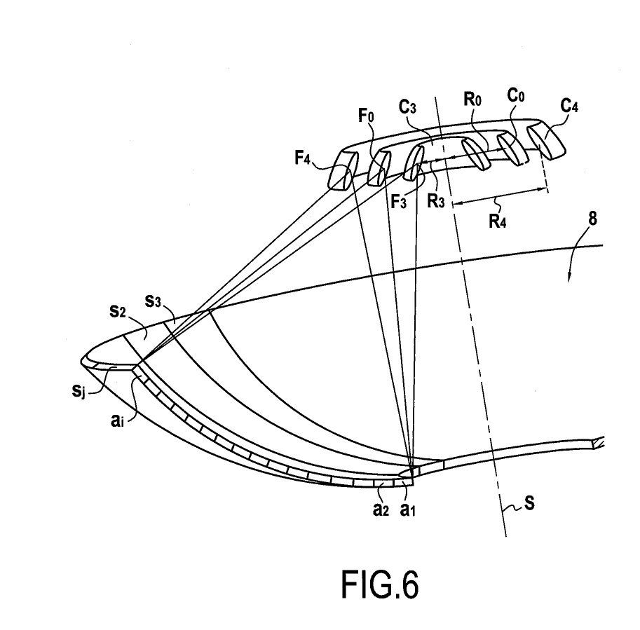

FIG. 6 illustrates another variant embodiment of geometric distribution of the ultrasound emitters 3. In the example illustrated in FIG. 6, the ultrasound emitters 3 are distributed into concentric rings a₁ to aᵢ, as illustrated in FIG. 5, but are also divided into radial sectors s₁ to sⱼ. In other terms, the emission face 8 of the transducer is cut out by planes passing through the axis of symmetry S so as to produce a succession of radial sectors. The decomposition of the transducer into annular and radial ultrasound emitters uses electronic focusing means to shift the focal crown from its natural position C₀ to lateral positions C₃ and C₄. Each focal crown C₀, C₃, C₄ is positioned in the space by the focal distance respectively F₀, F₃, F₄ separating it from the transducer and by its diameter respectively R₀, R₃, and R₄. The focusing crown is thus in the same plane parallel to the upper plane of the transducer which is perpendicular to the axis of symmetry S. It should be noted that the combination of the electronic focusing means utilised for the configurations described in FIGS. 5 and 6 modifies the incidence of this plane relative to the upper plane of the transducer, but also makes deformed crowns according to complex surfaces. This configuration adapts to treatment of tumors of different sizes.

It should also be noted that FIG. 6 illustrates distribution of the ultrasound emitters 3 according to concentric rings a₁ to aᵢ, divided into radial sectors s₁ to sⱼ. Of course, it can be provided that the emission face 8 of the transducer comprises a series of ultrasound emitters 3 only divided into radial sectors s₁ to sⱼ. Advantageously, the ultrasound emitters 3 of each of said radial sectors are activated by the control circuit 7 with a delay or phase law for ensuring shifting of the focusing crown.

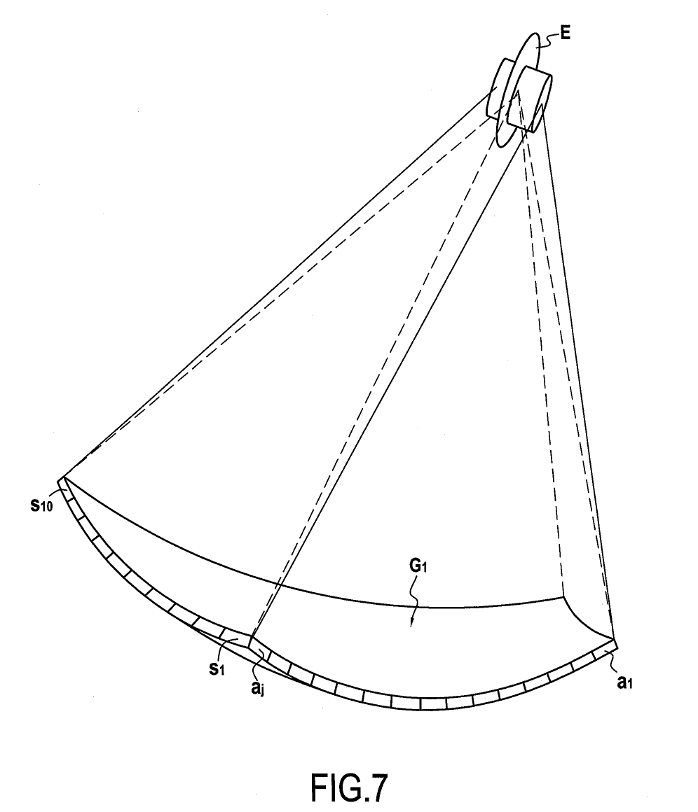

FIG. 7 illustrates another variant embodiment in which the radial sectors s₁ to sⱼ of ultrasound emitters are combined to form radial groups G₁ to Gₖ. Radial group should mean the combining of different radial sectors s₁ to sⱼ, such as defined in the example illustrated in FIG. 6 such that the ultrasound emitters 3 are distributed evenly in concentric rings a₁ to aᵢ. The radial sectors s₁ to sⱼ of the transducer are preferably combined identically to form successive identical groups G₁ to Gₖ. In the example illustrated in FIG. 7, each radial group G₁ to Gₖ comprises ten radial sectors s₁ to sⱼ. In addition, the transducer comprises for example ten radial groups G₁ to G₁₀ all comprising the same number of ultrasound emitters. In FIG. 7, only the radial group G₁ is illustrated.

The control circuit 7 is adapted to deliver signals to the ultrasound emitters of the radial groups as described hereinabove. The ultrasound emitters of each radial group are activated so as to concentrate the energy into a single focal volume. This focusing corresponds in the example illustrated to that obtained with a group of spherical shape even though the group has come from toric geometry. It is thus possible to concentrate the energy distributed on a focal crown in as many distinct focal volumes E as groups of ultrasound emitters G₁ to Gₖ. This concentration for example concentrates and reinforces tissue necrosis at one or more particular points of the tumor to be treated. It can also be envisaged to simultaneously form several local focal patches distributed on one focal crown.

According to another characteristic of the subject matter of the invention, the signal generator of the control circuit 7 is controlled to activate independently, alternatively and substantially consecutively the rings a₁ to aᵢ the radial sectors s₁ to sⱼ or the radial groups of ultrasound emitters G₁ to Gₖ such as described hereinabove in FIGS. 5 and 6, to ensure continuous insonification originating respectively from rings, sectors or different radial groups. It must be understood that the subject matter of the invention is to independently, alternatively and substantially consecutively activate the ultrasound emitters organised into several categories N₁ to Nₘ, each of the categories associating or combining either one or more concentric rings of ultrasound emitters, or one or more radial sectors of ultrasound emitters, or one or more radial groups of ultrasound emitters.

As emerges more clearly from FIG. 8A to 8C, the ultrasound emitters of the category N₁ are activated to realise a shot T₁. At the end of the duration of the shot T₁, the ultrasound emitters of the category N₁ are no longer activated to let the ultrasound emitters and the intermediary tissue situated between the ultrasound emitters of the category N₁ and the focusing zone cool down. On completion of the shot T₁ of the category N₁, the ultrasound emitters of the category N₂ are activated so as to complete a shot T₂ over a duration for example equal to the duration of the first shot. The shot T₂ is interrupted to let the ultrasound emitters and the intermediary tissue situated between the ultrasound emitters of the category N₂ and the focusing zone cool down. The control circuit 7 continues consecutive activation of the categories of ultrasound emitters N₁ to Nₘ. The categories of ultrasound emitters N₁ to Nₘ are thus activated one after the other to complete shots respectively T₁-Tₖₘ₊₁ . . . ; T₂-Tₖₘ₊₂ . . . , with k=1, 2 . . . in focusing volumes different to one another and placed substantially contiguously.

Contrary to dynamic focusing methods, it is to be considered that the ultrasound emitters of a category are activated independently and substantially consecutively from the ultrasound emitters of the other category. As is evident from the preceding description, the categories of ultrasound emitters N₁ to Nₘ are activated alternatively, that is, successively or one after the other. As will be described in greater detail following the description, the categories of ultrasound emitters N₁ to Nₘ are activated according to an order or a determined sequence, variable or not.

Advantageously and ideally, the ultrasound shot T₂ of the category N₂ is realised immediately following the ultrasound shot T₁ of the category N₁ without delay or advance. In practice, it must be considered that the control circuit 7 substantially consecutively activates the categories of the ultrasound emitters so as to ensure continuous insonification in the focusing zone. In fact, it proves that the instrumentation employed can in practice cause discontinuity or recovery between the shots. It is considered that a shot T₂ of a category N₂ is substantially consecutive to a shot T₁ of a category N₁ if the delay time taken between the end of the shot T₁ and the start of the shot T₂ is not sufficient to lose the benefit of the cavitation and thermal heating activities of the preceding shots. Similarly, two shots T₁ and T₂ are considered substantially consecutive if the recovery time between these shots is sufficiently short not to produce burning of the tissue.

As is evident from FIG. 8D, the treatment zone is insonified continuously in turn by a category N₁ to Nₘ. Ultrasound treatment is thus carried out continuously without stressing the same ultrasound emitters. The time between two ultrasounds shots is zero or sufficiently short for cavitation and thermal heating activities to be maintained at the level of the treatment zone. Each ultrasound shot thus benefits immediately from the rise in temperature and the rate of bubbles created by the preceding shot without having to reform them between two shots. This technique reinforces the volume of each lesion associated with each category of ultrasound emitters and/or considerably decreases treatment time.

Distribution of the ultrasound emitters into different categories continuously applies on the same focal volume the thermal dose necessary for destruction of this volume from several distinct categories excited sequentially and each acting in an independent and substantially contiguous focal volume.

It is thus possible to continuously treat lesions having a large volume, without stressing the same elements at each ultrasound shot. To the extent where each category contains several ultrasound emitters, it is possible to combine this treatment principle with electronic focusing, as described hereinabove. The ultrasound emitters of each category are then activated with a delay or phase law for ensuring electronic shifting of the treatment zone. It must be understood that this delay or phase law is applied solely for the ultrasound emitters belonging to the same category. In other terms, there is no electronic focusing between the different categories. Of course, it can be feasible to ensure mechanical shifting of the ultrasound emitters.

Although the target zone is heated continuously, the tissue located between the ultrasound emitters and the treatment zone receives ultrasounds only sequentially, which produces their cooling. In fact, the tissue located on the acoustic path is exposed to the ultrasound emitted by each category solely when the latter is activated. Also, reheating the ultrasound emitters of each category is controlled by their sequential activation. The ultrasound emitters of a category are cooled during activation of a following category.

The ultrasound emitters can be activated in different ways. Therefore, the control circuit 7 can deliver signals to activate the categories of ultrasound emitters according to a sequence which repeats cyclically. For example, the categories N₁, N₂, N₃, N₄ are activated one after the other, and this activation sequence N₁, N₂, N₃, N₄ is repeated over time.

Naturally, it can be provided that the activation sequence varies over time. For example, during the first sequence, the categories N₁ to N₄ are sequentially activated in the order N₁ to N₄, whereas during the following sequence, the categories are activated sequentially in the following order: N₁, N₃, N₂, N₄.

According to another possible variant, the control circuit 7 delivers signals to activate assemblies of categories of ultrasound emitters composed of distinct categories of ultrasound emitters. Therefore, sequential group activation can be envisaged for the categories for example N₁-N₂, N₃-N₄. In other words, the ultrasound emitters of the categories N₁ and N₂ are activated simultaneously, then those of the categories N₃-N₄. This sequential group activation can be done cyclically (N₁-N₂, N₃-N₄, N₁-N₂, N₃-N₄ . . . ) or be modified in the following form, for example (N₁-N₂, N₃-N₄, N₁-N₄, N₂-N₃ . . . ).

According to another variant embodiment, the control circuit 7 delivers signals to activate assemblies of categories of ultrasound emitters composed of categories, at least some of which are common to all assemblies. Such a variant is advantageously employed by a transducer comprising a large number of categories Ni. Group activation of the categories N₁-N₂-N₃-N₄ can thus be envisaged, then of categories N₄-N₅-N₆-N₇, then of groups N₇-N₈-N₉-N₁₀, etc. According to this example, there is recovery of the categories equal to 25%. The recovery surface must be sufficiently small so as not to produce, during consecutive shots, burning of the tissue located between the ultrasound emitters of the recovery zones and the focusing zone.

The subject matter of the invention is applied particularly advantageously in all treatments by focused ultrasound, in particular treatment of cancer of the prostate and treatment of liver tumors.

According to another advantageous characteristic, the subject matter of the invention aims at decreasing the number of coaxial connection cables between the ultrasound emitters 3 and the control circuit 7. To reduce the number of cables it is proposed to execute switching of the electrical grounds of the ultrasound emitters 3 in order to substantially decrease the number of coaxial cables necessary, as well as the need for control electronics (signal generator and power amplifier).

As is more evident from FIG. 9, the principle is to distribute the ultrasound emitters evenly and identically over the radial groups G₁ to G_{K}. Each radial group G₁ to G_{K} therefore comprises the same number i of ultrasound emitters 3. All the ultrasound emitters bearing the same number in each radial group are identical and the cores 100 of the coaxial cables of all these ultrasound emitters of the same row are all connected together. The grounds 101 of the coaxial cables of all the transducers of the same radial group are connected together. There are as many grounds as radial groups. The grounds 103 of the radial groups not utilised during an ultrasound shot are not connected to the ground 104 of the excitation signal delivered for the control circuit 7. The grounds of the active radial groups are connected to the ground 104 of the excitation signal. Therefore, the excitation signal of the ultrasound emitters will be converted into acoustic energy only for the transducers of the radial groups whereof the grounds are physically connected to the ground of the excitation signal. Switching from one ground to the other can be done using demultiplexers 105 controlled via a command 106 by the control circuit 7. By way of example, for a transducer containing 256 ultrasound emitters distributed equally over 8 radial groups, it is normally necessary to use one coaxial cable per transducer and thus connect 257 wires. As per the commutation principle by grounds, the number of cables is 40 wires.

The invention is not limited to the examples described and illustrated, as various modifications can be made without departing from its scope.

1. Therapy apparatus for the treatment of tissue by the emission of focused ultrasound waves, characterised in that it comprises:
a probe (1) comprising a transducer (2) formed by ultrasound elements (3) distributed into several categories (N1 to NM) on an emission face of focused ultrasound waves (8) which is a revolution surface engendered by the rotation of a segment of a curve about an axis of symmetry, the categories of ultrasound elements defining independent and substantially contiguous focal volumes,
and a control circuit (7) comprising a signal generator controlled to activate independently, alternatively and substantially consecutively the categories of ultrasound emitters so as to ensure continuous insonification according to a crown originating from different categories of ultrasound emitters.

2. Therapy apparatus as claimed in claim 1, characterised in that the ultrasound elements are distributed over an emission face of focused ultrasound waves (8) having a revolution surface engendered by rotation about an axis of symmetry (S), of a segment of a curve concave or convex of length (I) having a centre of curve (C) located at a distance (R) from the axis of symmetry (S), with R≠0.

3. Therapy apparatus as claimed in claim 2, characterised in that the revolution surface is engendered by a segment of an arc of a circle of length (I), of radius (r) and with a centre (c) located at a distance (R) from the axis of symmetry with R≠0.

4. Therapy apparatus as claimed in claim 2, characterised in that the distance (R) is less than the radius (r).

5. A therapy probe as claimed in claim 1, characterised in that the revolution surface delimits in its central region an opening (11) centered on the axis of symmetry (S) and adapted to receive an imaging transducer.

6. Therapy apparatus as claimed in claim 1, characterised in that the ultrasound emitters (3) are distributed according to concentric rings (a₁ to aᵢ) and in that the control circuit (7) comprises a signal generator controlled to deliver signals to activate the ultrasound emitters of each of said concentric rings, with a delay or phase law for ensuring shifting of the focusing crown according to the focusing axis (A).

7. Therapy apparatus as claimed in claim 1, characterised in that the ultrasound emitters (3) are distributed into radial sectors (s₁ to sⱼ) and in that the control circuit (7) comprises a signal generator controlled to deliver signals to activate the ultrasound emitters (3) of each of said radial sectors, with a delay or phase law for ensuring shifting of the focusing crown.

8. Therapy apparatus as claimed in claim 1, characterised in that the ultrasound emitters (3) are distributed into concentric rings (a₁ to aᵢ), divided into radial sectors (s₁ to sⱼ) and in that the control circuit (7) comprises a signal generator controlled to deliver signals to activate the ultrasound emitters (3) of each of said radial sectors, with a delay or phase law for ensuring shifting of the focusing crown.

9. Therapy apparatus as claimed in claim 8, characterised in that the radial sectors of ultrasound emitters (s₁ to sⱼ) are combined to form radial groups (G₁ to Gₖ) and in that the control circuit (7) comprises a signal generator controlled to deliver signals to activate the ultrasound emitters of each of said radial groups.

10. Therapy apparatus as claimed in claim 9, characterised in that the control circuit (7) comprises a signal generator controlled to deliver for each radial group (G₁ to G_{K}), signals to activate the ultrasound emitters of each of the radial groups, with a delay or phase law.

11. Therapy apparatus as claimed in claim 1, characterised in that the control circuit (7) comprises a signal generator controlled to deliver signals to activate, according to a sequence which varies or which repeats cyclically, a category of ultrasound emitters.

12. Therapy apparatus as claimed in claim 11, characterised in that the control circuit (7) comprises a signal generator controlled to deliver signals to activate assemblies of categories of ultrasound emitters composed either of distinct categories of ultrasound emitters or of categories whereof at least some are common to the assemblies.

13. Therapy apparatus as claimed in claim 9, characterised in that:
the radial groups (G₁ to G_{K}) form identical assemblies of ultrasound emitters,
the control circuit (7) is connected to the ultrasound emitters by means of coaxial cables (5), whereof on the one hand, the cores of all the ultrasound emitters of the same row in the different radial groups are connected together and on the other hand the grounds of all the ultrasound emitters of the same radial group are connected to one another, the signal to activate the ultrasound emitters being converted into ultrasound energy solely by the ultrasound emitters of the radial group(s) whereof the grounds are physically connected to the ground of the activation signal.
